(19) United States
(12) Patent Application Publication
(10) Pub. No.: US 2025/0369923 A1
(43) Pub. Date: Dec. 4, 2025

(54) SYSTEM AND METHOD FOR DETERMINING THE VISCOSITY AND ELASTICITY OF HYDROGELS USING ULTRASOUND

(71) Applicant: THE GEORGE WASHINGTON UNIVERSITY, Washington, DC (US)

(72) Inventors: Megan Jariwala, Flagstaff, AZ (US); Kausik Sarkar, Ashburn, VA (US); Kartik Venkat Bulusu, Vienna, VA (US); Michael W. Plesniak, Accokeek, MD (US); Lijie Zhang, Ashburn, VA (US)

(73) Assignee: THE GEORGE WASHINGTON UNIVERSITY, Washington, DC (US)

(21) Appl. No.: 19/225,632

(22) Filed: Jun. 2, 2025

Related U.S. Application Data
(60) Provisional application No. 63/654,570, filed on May 31, 2024.

(51) Int. Cl. G01N 29/07; G01N 11/00; G01N 29/28; G01N 33/483

(52) U.S. Cl. CPC G01N 29/07; G01N 11/00; G01N 29/28; G01N 33/4833; G01N 2011/0073; G01N 2291/011; G01N 2291/023; G01N 2291/02827; G01N 2291/101

(57) ABSTRACT

A system for analyzing a hydrogel sample. The system has a plate configured to retain the hydrogel sample, an ultrasound transmitter configured to propagate an ultrasound signal toward the hydrogel sample retained at said plate, and an ultrasound detector configured to receive a reflected ultrasound signal reflected by said plate and passing through the hydrogel sample. A processing device determines a property of the hydrogel sample based on the reflected ultrasound signal, wherein the property comprises viscosity. The processing device can then use the determined property to identify the hydrogel, or compare it with other materials.

### RELATED APPLICATION

This application claims the benefit of priority of U.S. Provisional Application No. 63/654,570, filed May 31, 2024, the entire content of which is relied upon and incorporated herein by reference in its entirety.

The following dissertation is incorporated herein by reference in its entirety: Acoustic and Rheological Characterization of Hydrogel Viscoelasticity by Megan Anderson Jariwala, The George Washington University.

### GOVERNMENT LICENSE RIGHTS

This invention was made with Government support under Grant/Contract No. CBET 2037849 awarded by the National Science Foundation (NSF). The Government has certain rights in this invention.

### BACKGROUND

Hydrogels mimic native soft tissues which has made them popular for a range of biomedical applications. Given that native tissues are complex viscoelastic materials, viscoelastic characterization of hydrogels is crucial for targeting specific needs. Traditional characterization methods utilize mechanical and rheological tests, requiring specific sample sizes, extensive sample preparation, and destructive measurements. Acoustic characterization techniques offer promising alternatives, but they rarely include insight into viscosity and often require complicated equipment or materials that behave as liquids.

Hydrogels exhibit complex viscoelastic behaviors which are difficult to quantify. Shear rheology, as shown in FIG. 1(a), is a standard technique for obtaining both the viscosity and elasticity of hydrogels, but it comes with experimental challenges such as instrument sensitivity, slip between the sample surface and the rheometer plates, and effects of gap height on the sample. Furthermore, shear rheology does not work for hydrogels that fracture at high shear rates or degrade at high temperatures, making the use of time-temperature superposition inapplicable in some cases. To avoid these limitations, shear rheology experiments on hydrogels tend to span low shear rates and frequencies. Additionally, hydrogels are often tested before undergoing full chemical crosslinking because the results are relatively easy to obtain and can be used to inform biomedical applications relating to 3D-printing and injectable materials. Typical shear flow sweeps of hydrogels, used to obtain hydrogel viscosity as a function of shear rate, are shown in FIG. 1(b).

Even when a full viscoelastic characterization has been performed on a hydrogel using shear rheology, it is often supplemented with compression testing, as shown in FIG. 1(c). Unlike shear rheology, compression testing requires samples that are elastic enough to hold their shape, i.e., viscoelastic solids such as crosslinked hydrogels. It results in stress-strain curves, illustrated in FIG. 1(d), which are used to calculate the elastic modulus of the hydrogel. Though traditional stress-strain curves cannot provide insight on a hydrogel's viscosity, the elastic modulus can easily be compared to other values in the literature. This is particularly relevant for tissue engineering applications, where hydrogel stiffness has been identified as an important characteristic for cell growth and differentiation. Shear rheology and compression analysis are destructive to the sample.

### SUMMARY

The present disclosure provides a pulse echo ultrasound system to obtain both the viscosity and elasticity of a hydrogel, resulting in a non-invasive viscoelastic characterization. We performed our characterization on polymeric hydrogels of various concentrations, types, and curing times to gain insight on how viscoelasticity is affected by different parameters. The results closely align with results from traditional testing methods, demonstrating the effectiveness of this acoustic method. As used here, a hydrogel (or polymetric hydrogel) is a polymer with a high amount of water, and has some viscosity (greater than zero). In some embodiments, at least 10% water by volume to be classified as hydrogel. In some embodiments, the hydrogel can be a biomaterial sample.

### BRIEF DESCRIPTION OF THE DRAWINGS

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIGS. 1(a)-(h) show viscoelasticity characterization systems, methods and illustrative experimental data. The data shown are for GelMA hydrogel samples (60 s curing time) of varying concentrations: 10% (yellow), 15% (red), and 20% (blue).

FIG. 1(a) shows a rotational rheometer system having a hydrogel sample between stainless steel crosshatched plates.

FIG. 1(b) is a graph of flow sweeps obtained using the rotational rheometer of FIG. 1(a). The viscosity of the samples is shown as a function of shear rate. Shear thinning is exhibited by all three samples.

FIG. 1(c) is a compression testing setup showing a hydrogel sample as it is uniaxially compressed.

FIG. 1(d) is a graph showing stress-strain curves obtained from the compression test. Young's modulus can be found as the slope of the linear region of each curve.

FIG. 1(e) is an acoustic system for the pulse echo ultrasound tests. A transducer (2.25 MHz center frequency) is submerged in a chamber filled with water. A hydrogel sample is at the bottom of the chamber.

FIG. 1(f) is a graph of an example of the signals received by the transducer. The test signal (which traveled through the water and the hydrogel) arrives earlier and has a smaller amplitude than the control signal (which traveled through the water alone) due to sound traveling faster and attenuating more in the hydrogel.

FIG. 1(g) is a graph showing acoustic attenuation coefficients for 1.25-3.75 MHz, calculated using the difference in amplitude between the control and test signals. Using these values, the viscosity of each sample can be calculated as a function of frequency.

FIG. 1(h) is a graph showing speed of sound through each hydrogel. These values can be used to calculate the bulk moduli.

FIG. 1(i) is an enlarged view of FIG. 1(e).

FIG. 1(j) is an enlarged view of FIG. 1(f).

FIG. 2(a) is a graph showing elastic moduli of the samples. The bulk modulus of each sample was calculated using hydrogel sound speeds from pulse echo ultrasound tests.

FIG. 2(b) is a graph showing elastic moduli of the samples. Young's modulus of each sample was calculated from compression tests

FIGS. 3(a)-3(j) are graphs illustrating a comparison of the viscosity values measured using rotational rheometry (blue) and the viscosity values calculated from pulse echo ultrasound (red). Individual viscosity values with their standard deviations are plotted at their corresponding frequencies. The average of the measured viscosities (dashed blue line) and the average of the calculated viscosities (dashed red line) are also plotted.

FIGS. 3(a), 3(b), and 3(c) show how both viscosities increase with GelMA concentration.

FIGS. 3(d), 3(e), and 3(f) show how both viscosities increase with the addition of GelMA to PEGDA hydrogels.

FIGS. 3(g), 3(h), and 3(i) show how both viscosities increase with curing time between 30 s and 90 s.

FIG. 3(j) shows how both viscosities decrease between 90 s and 120 s curing time.

FIGS. 4(a)-(c) are graphs showing a comparison of the viscosity trends measured using rotational rheometry (blue) and the viscosity values calculated from pulse echo ultrasound (red).

FIG. 4(a) shows the viscosity increased with each increase in GelMA concentration, and the pulse echo ultrasound values fall within the standard deviation of the rotational rheometry values.

FIG. 4(b) shows the viscosity increased as GelMA was added to PEGDA hydrogels, with the pulse echo ultrasound values closely following the rotational rheometry values.

FIG. 4(c) shows the viscosity increased as the curing time of the GelMA hydrogels increased from 30 s to 90 s, then the viscosity decreased as the curing time increased from 90 s to 120 s. The standard deviations of the viscosities from rotational rheometry and pulse echo ultrasound overlap.

FIG. 5(a) is a schematic of the acoustic system showing the ultrasound signal traveling through water and a hydrogel. The distance between the transducer face and the bottom of the acoustic testing chamber is l.

FIG. 5(b) is an enlarged view of the chamber of FIG. 5(a).

FIG. 6 is a flow diagram of the acoustic system.

FIG. 7 is a flow diagram of obtaining viscosity for a sample.

FIG. 8 is a flow diagram of obtaining elasticity for a sample.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

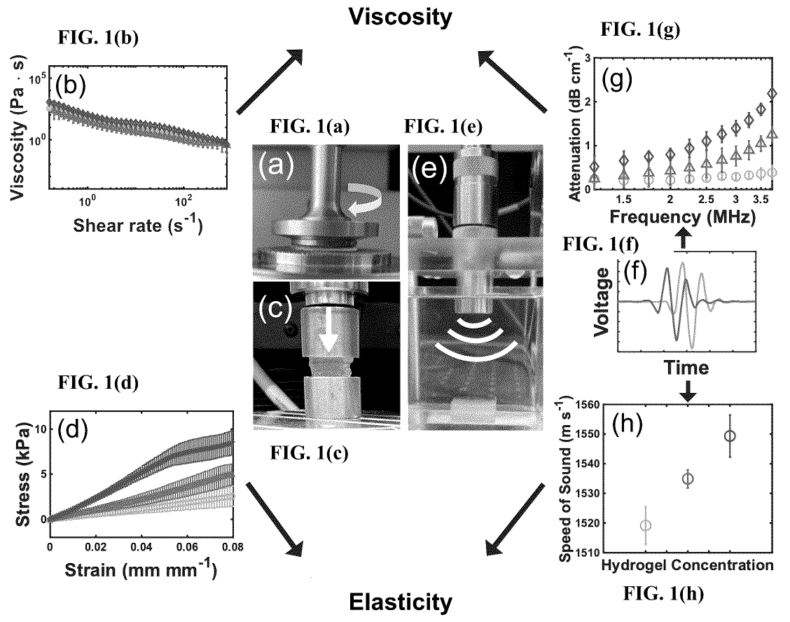

Turning to the drawings, FIGS. 1(e), 1(i) show a detection and characterization system 100 in accordance with one non-limiting illustrative embodiment of the disclosure. The characterization system 100 provides pulse echo ultrasound as an alternate technique for the viscoelastic characterization of hydrogels.

The characterization system or assembly 100 includes a housing 110, container 120, transmission medium 130, and transducer 150. As shown, the container 120 has one or more side walls, a closed bottom and an open top, which defines a container interior and a container exterior. The transmission medium 120 is a liquid, such as water, and is retained in the container interior of the container 120. In other embodiments, the transmission medium can be an ultrasound gel or the like. A sample 10 is placed in the transmission medium 120 at the bottom of the container 120. The housing 110 is arranged with respect to the container 120 to position the transducer 150 with respect to the container 120, transmission medium 120, and the sample 10. The housing 110 can include a platform that is above the open top of the container 120.

In the example embodiment shown, the transducer 150 can be elongated with a transducer longitudinal axis, a transducer proximal end and a transducer distal end. The transducer 150 has a transducer transmitter and a transducer detector, both located at a distalmost end of the transducer distal end. The transducer emits a ultrasound wave from the transducer transmitter at the distalmost end of the transducer distal end. The transducer 150 is coupled to the housing 110, such as at the platform. The transducer 150 extends substantially perpendicular to the top of the container 120 and the top surface of the transmission medium 130, and the top surface of the sample 10. The transducer proximal end is at the container exterior, and the transducer distal end is at the container interior. The distalmost end of the transducer distal end is at least partly positioned or submerged in the transmission medium 130. Accordingly, the distalmost end of the transducer distal end is approximately orthogonal to and faces the sample 10. The ultrasound wave is emitted from the distalmost end of the transducer distal end toward the sample 10. The ultrasound wave travels through the medium 130 and strikes the sample 10. The wave bounces off of the sample 10, which in the current embodiment is a hydrogel, and returns to a wave detector at the transducer.

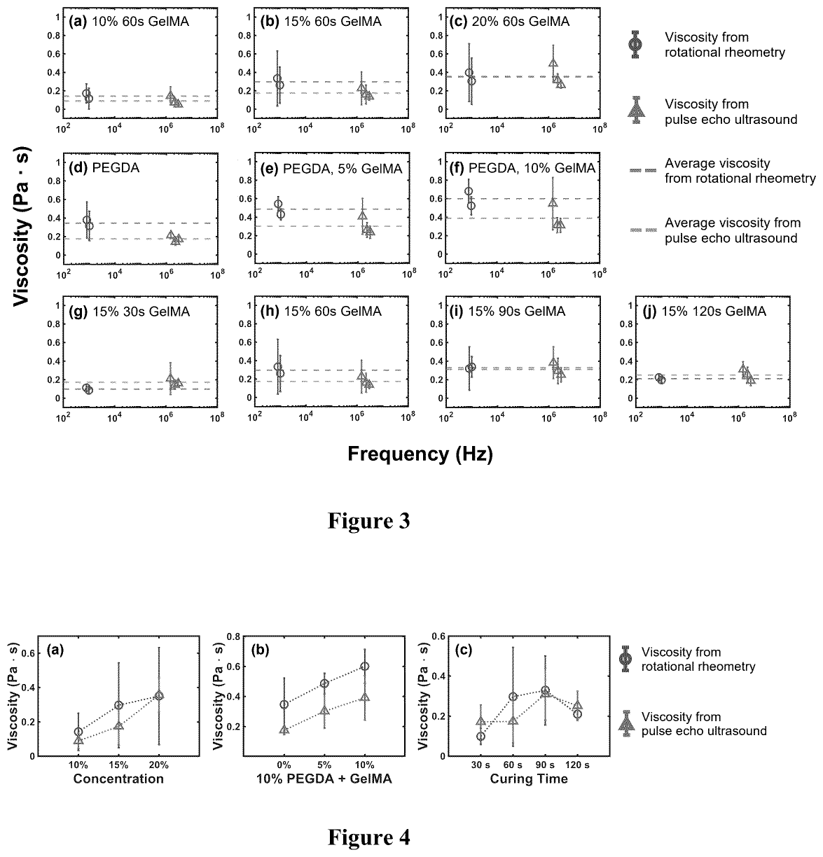

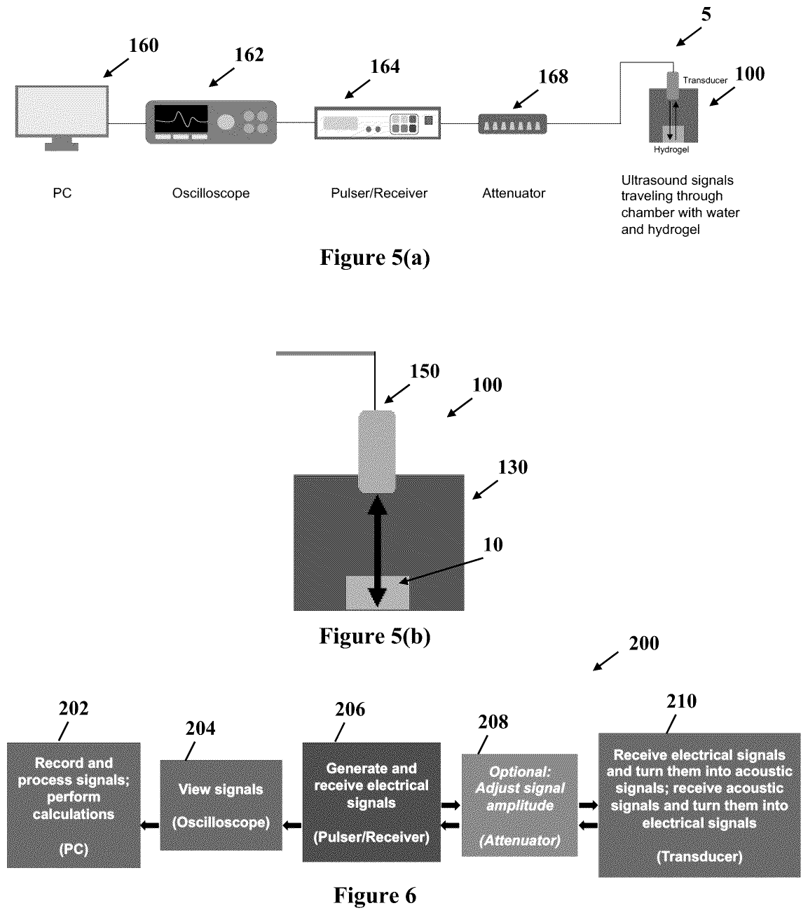

FIGS. 5(a), (b) are a schematic of the overall system 5, and FIG. 6 is a flow chart including the overall operation of each component in the overall system 5. The overall system 5 includes for example a processing device 160, such as a personal computer (PC), an oscilloscope 162, pulser/receiver 164, attenuator 168, and a transducer 150. Starting at step 206, the pulser/receiver 164 generates a signal. At step 208, the optional attenuator 168 adjusts the electrical signal pulse from the pulser 164, and passes the attenuated signal to the transducer 150. Then at step 210, the electrical signals are received at the transducer 150, which turns them into acoustic signals and transmits the acoustic signals at the transducer transmitter located at the transducer distal end. The hydrogel is shown contained in a container and surrounded by a liquid such as water. The transducer 150 is in contact with the liquid, so that ultrasound signals from the pulser or attenuator can be transmitted to the hydrogel. The signal is reflected by the hydrogel sample 10 and the reflected signal is received by the transducer detector. The transducer 150 turns the reflected signals back into electrical signals. The reflected electrical signal is sent to the oscilloscope 162 to be viewed, step 204, and then to the PC 160 to record and process the signals and perform calculations, step 202.

FIGS. 5(a), 5(b) show the processor 160, transducer 150, user interface and sample holding chamber 110 as separate elements. In other embodiments, the processor, transducer, user interface, and sample holding chamber are combined into a single, portable measurement device that may be connected to a power source. In addition, a user interface may be used to input sample values (such as density) into the processor. In certain embodiments, the system may be further configured to measure the density of the sample (such as by weighing the sample to get the mass and scanning the sample to get the volume). In some embodiments, the processor 160 may also be configured to calculate the acoustic properties of the sample then calculate the sample viscosity, with these results appearing on the user interface. In some embodiments, the user interface could be a screen on the system, a PC connected to the system, or a cell phone wirelessly connected to the system. In some embodiments, acoustic frequencies from 1.5-3 MHz work well for this application, though other ranges can be provided such as 0.5-5 MHz. In addition to hydrogels, the system can be utilized for biological tissues and other materials with high shear rate (˜750 s-1 and above) viscosities between 0 and 1 Pa s. The range of ultrasound excitation (1-5 MHz frequency and 15-40 kPa amplitude) used here is safe for biological applications.

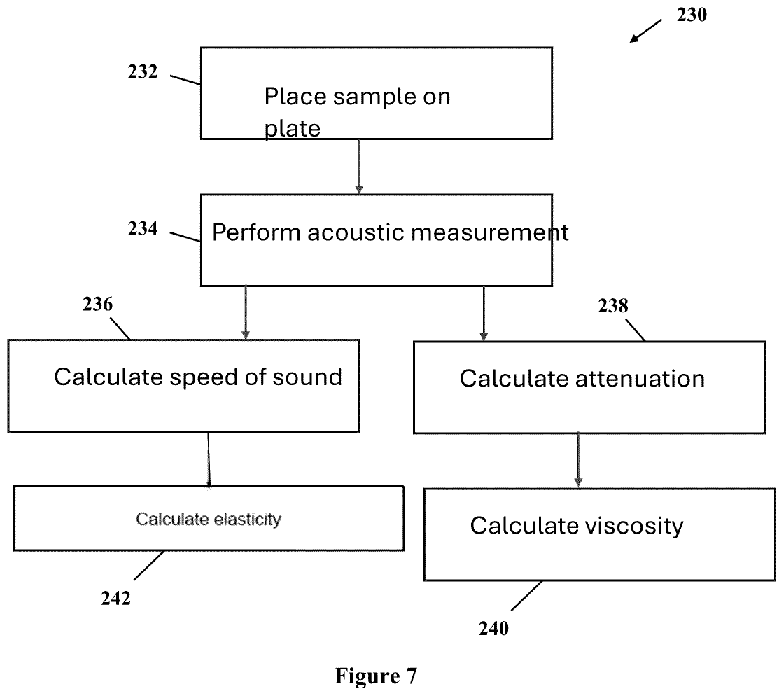

FIG. 7 shows the overall operation 230 of the computer 160 to calculate the speed of sound, acoustic attenuation, and viscosity, step 202. Starting at step 232, the sample 10 is placed in a container 120, such as a plate. At step 234, the transducer 150 is operated by the computer 160, step 202, pulser 164, and the acoustic measurement is received, step 206. The acoustic measurement 234 is the measurement of the reflected signal generated by the transmitted excitation, and represents how the reflection is affected by the sample (e.g., the raw data is shown in FIG. 1(j)). The oscilloscope 12, pulser/receiver 164, attenuator 168 are utilized to generate and process the signal and receive and process the reflected signal. At this point, the computer can calculate the speed of sound 236 through the hydrogel sample 10 which is a property of the hydrogel material. The speed of sound, step 236, is calculated based on the transmitted signal and the reflected signal (FIG. 1(j)), which is the acoustic signal from step 234. The speed of sound can then be utilized to calculate elasticity, step 242, which is one of the material properties of the hydrogel 10, as shown for example with respect to the description of Equation (3) below for speed of sound c (there cₕ) and Equation (1) for elasticity K.

The computer 160 also calculates the acoustic attenuation of the hydrogel sample 10. The same acoustic measurement data, step 234, used to determine the speed of sound (step 236) is also used to determine the acoustic attenuation, step 238, i.e. the raw acoustic measurement data shown in FIG. 1(j). The acoustic attenuation, step 238, is used to determine viscosity of the hydrogel sample, step 240. The computer 160 calculates the viscosity of the sample 10. Calculating attenuation, step 238, is used to calculate the viscosity, step 240. The speed of sound through the sample (236), acoustic attenuation (238) and/or viscosity (240) can then be used, for example, to compare to properties of tissues or substrates and match to hydrogels. Thus, the system 5 provides a non-invasive, non-destructive material testing to determine viscosity and elasticity of a hydrogel sample. The transmission medium is the only contact made with the sample. The transducer does not contact the sample 10 and the ultrasound signals do not affect the sample.

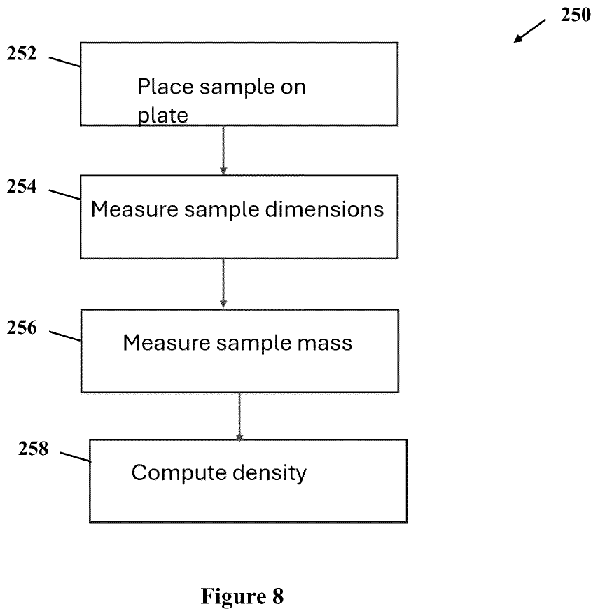

FIG. 8 flowchart shows additional method steps 254, 256, 258. Here, it is shown that after the sample 10 is placed on the plate, step 252, the system can obtain the dimensions of the sample 10, step 254, including for example length, width, and height of the sample, which is certain embodiments is a cuboid shape. In one or more embodiments, the sample dimension measurement may be taken while the sample is in the system (using, for example, an optical measurement device, laser measure, or calipers). The sample thickness can be used, for example, for the speed of sound and attenuation calculations, steps 236, 238 (FIG. 7). It also is used to determine the mass of the sample 10, step 256, and the acoustic measurement, step 234, and the computer 160 then calculates the density, step 258, of the sample 10 based on the weight and dimensions, steps 254, 256.

As in FIG. 7, the computer 160 calculates the speed of sound, step 236, acoustic attenuation 238, and viscosity 240. And at step 242, the computer 160 calculates elasticity of the sample 10 based on the acoustic measurement data shown in FIG. 1(j). The acoustic measurement data, step 258, is used to calculate the speed of sound and attenuation, steps 236, 238. See Eq. (3) for speed of sound. Eq (1) for elasticity. Eq. (2) for viscosity. The speed of sound is used to determine elasticity using density. The attenuation, step 238, is used along with density, step 258, to calculate viscosity, step 240. All the acoustic data 234 comes from the transducer. Sample elasticity affects viscosity 240, speed of sound 236, acoustic measurement 234 and density 258, though doesn't affect acoustic attenuation 238. The processor 160 can then use viscosity and/or elasticity (with any one or more of the measured or calculated values including density, speed of sound, and/or acoustic attenuation) to identify the hydrogel sample such as by comparing the sample with known hydrogels or hydrogel properties, or to determine a characteristic or type of the sample, such as for example a tissue; and the measure intrinsic properties of materials.

The acoustic measurement, step 234, must be performed without a sample 10 inside as a calibration measurement. This calibration measurement provides the control data needed for calculating attenuation. As with other standard system calibrations, the calibration measurement does not need to be performed every time the system is used. Rather, it needs to be performed before the first use of the system and any time a system variable changes (e.g., if the distance between the transducer face and the plate changes, if water replaces ultrasound gel, etc.).

Pulse echo ultrasound, shown in FIG. 1(i), is performed by sending an ultrasonic pulse out of the transducer 150 and recording the echo of that pulse when it returns to the transducer detector. By comparing the echoes, as shown in FIG. 1(f), 1(j), with and without a hydrogel sample 10 in the signal's path, the hydrogel's attenuation coefficients, shown in FIG. 1(g) (step 238; FIGS. 7, 8), and speed of sound, shown in FIG. 1(h) (step 236), are determined. The speed of sound c (step 236) through a material can be paired with the material's density ρ (step 258) to calculate the bulk modulus K [1]:

$$K = ρ {c}^{2} . \qquad ( 1 )$$

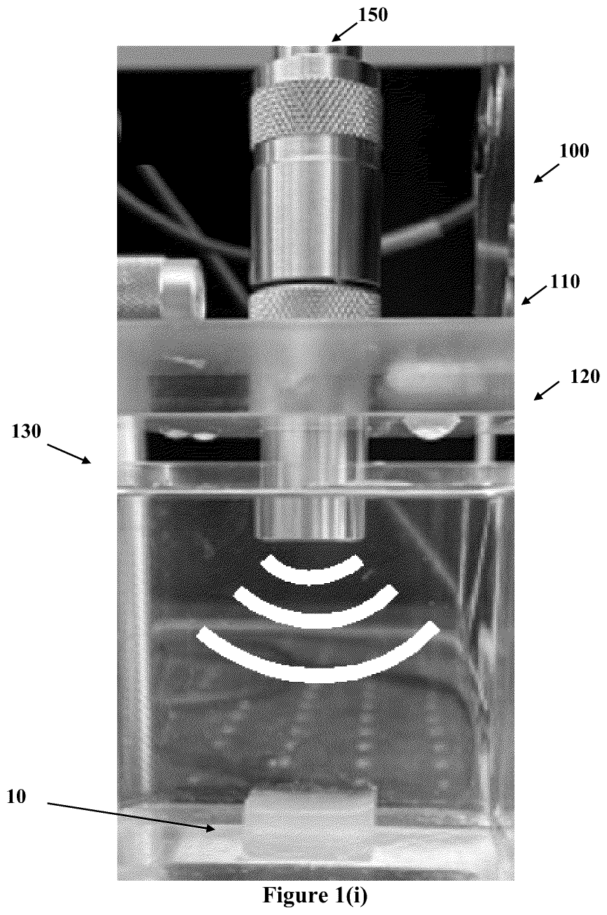

We performed this elastic characterization for gelatin methacrylate (GelMA) and poly-(ethylene glycol)-diacrylate (PEGDA) hydrogels, two popular biomaterials used in the field of tissue engineering. All hydrogels were chemically crosslinked (cured) by pipetting them into a mold, layer by layer, and exposing each layer to ultraviolet (UV) light for a set amount of time. The bulk moduli of these hydrogels are shown in FIG. 2(a). They range from 2.3 to 2.6 GPa and increase with both hydrogel concentration and the addition of GelMA in PEGDA hydrogels. Additionally, they increase from 30 s to 90 s curing time then appear to plateau.

For comparison, we performed compression testing to obtain the Young's moduli for our sample set. These values are shown in FIG. 2(b) and range from 25 to 150 kPa. Notably, they show the same trends as those seen in the bulk moduli, using a completely different means, i.e. speed of sound. The elasticity values are very sensitive to Poisson's ratio which prevents direct comparison between the bulk modulus and Young's modulus. An advantage of the bulk moduli measurements is that, unlike compression testing, it can be performed on living human tissues.

To characterize the viscosity of our sample set using ultrasound, we used Stokes' law of sound attenuation,

$$α = \frac{2 {ηω}^{2}}{3 ρ {c}^{3}} , \qquad ( 2 )$$

where α is the attenuation coefficient at a specific angular frequency ω corresponding to dynamic viscosity η of the material. This equation was formulated for a plane wave traveling through a Newtonian fluid, and it predicts a linear relationship between signal loss (attenuation) and viscosity [4]. Later, the equation was modified to account for heat conduction, volume viscosity, and relaxation time [5, 6]. More recent work has used this equation to find the bulk viscosity of Newtonian fluids [7], expand the field of longitudinal rheology, and explore the theory needed to gain insight on more complex viscoelastic materials [8].

To the best of our knowledge, equation (2) has not previously been used to calculate the viscosity of a hydrogel. Since the equation was not formulated for this application, we made five assumptions. First, we assumed that the thermal conductivity and heat capacity values of our hydrogels closely approximated those of pure water [9-11]. This results in a negligible amount of attenuation due to heat conduction which can consequently be ignored. Second, we assumed that the volume viscosity of our samples would also result in a negligible contribution to the attenuation since hydrogels are nearly incompressible. Third, we assumed that viscosity, rather than molecular relaxation, was the dominant form of dissipation at our test frequencies. Fourth, since we did not add an acoustic scatterer to our samples, we assumed that scattering did not contribute to our measured attenuation. Finally, we assumed that our hydrogels are isotropic and homogeneous.

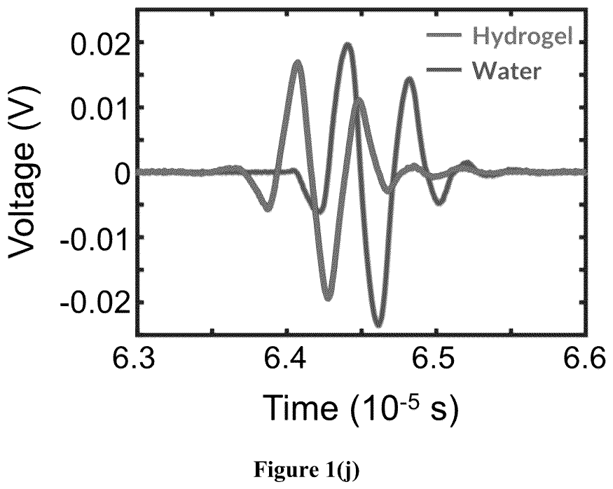

We calculated the viscosities of our samples using the attenuation coefficients at 1.5, 2.25, and 3 MHz. The results are shown in FIG. 3 with both the individual calculated viscosities and the average of the calculated viscosities plotted in red. The calculated viscosities range from 0.05 to 0.55 Pa s. As seen in the elasticity values, the calculated viscosities increase with both hydrogel concentration, FIGS. 3(a)-(c), and the addition of GelMA in PEGDA hydrogels, FIGS. 3(d)-(f). They also increase from 30 s to 90 s curing time, shown in FIGS. 3(g)-(i). At 120 s, however, there is a decrease in viscosity as seen in FIG. 3(j).

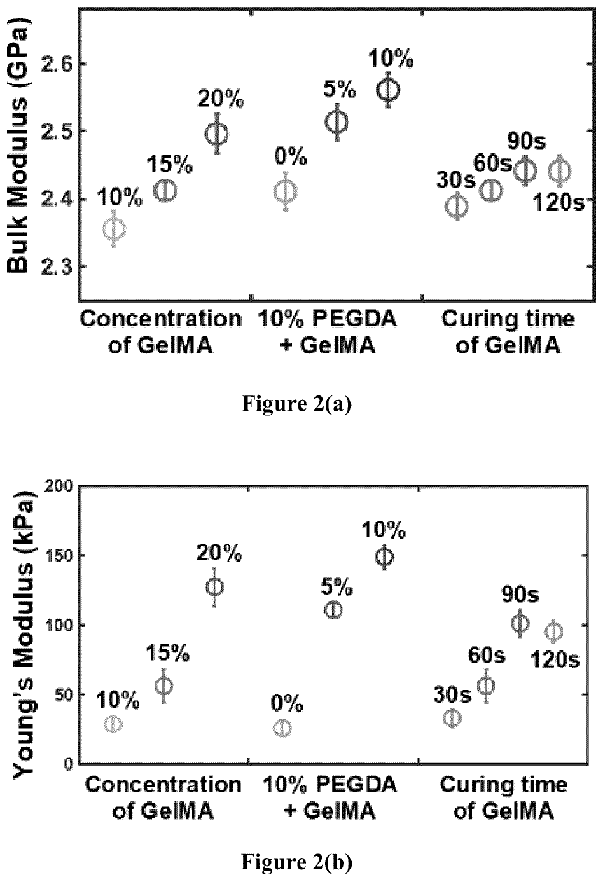

For a viscosity comparison, we performed flow sweeps on our sample set using a rotational rheometer. The measured viscosities decreased with shear rate, revealing shear thinning in the samples. There did not appear to be a viscosity plateau at either end of the curve, though the viscosity values at the two highest shear rates (794 and 1000 Hz) were a close match with each other. We selected these two high shear rate values for comparison with the viscosities calculated from acoustic attenuation. The viscosities measured from rheology range from 0.09 to 0.68 Pa s and are plotted in blue, both as individual values and as an average value, in FIG. 3. They are the same order of magnitude as the calculated viscosities and follow the same trends, even mirroring the non-monotonic behavior seen in the hydrogels with increasing curing times. These trends are highlighted in FIG. 4.

In conclusion, we developed an ultrasonic testing method for determining the viscoelasticity of a hydrogel. With a single ultrasound system, we are able to obtain both the high shear viscosity and the bulk modulus for each sample. One key advantage of our system and method over shear rheology and compression testing is the non-destructive nature of ultrasound; our method makes possible long-term viscoelastic analysis of a sample and provides a faster characterization method. This is particularly useful in biomedical applications where each sample can be time-intensive and expensive to fabricate. Furthermore, unlike other acoustic characterization techniques such as elastography and remote acoustic palpation [12, 13], our method produces quantitative results using a simple acoustic setup and minimal data analysis. Additionally, the results confirm our assumption that viscosity is the primary cause of attenuation in these hydrogels. Further investigation into viscoelastic dynamics of hydrogels using ultrasound can pave the way for more informed hydrogel selection and quality control in biomedical applications.

### Hydrogel Synthesis

GelMA was synthesized using an established procedure [14-16]. In summary, 5% (v/v) methacrylic anhydride (Sigma-Aldrich) was added dropwise to a solution of 10% (w/v) Gelatin (Type A, Gel Strength ˜300 Bloom, Sigma-Aldrich) dissolved in pure water. The solution was kept at 50° C. under magnetic stirring for two hours then dialyzed against pure water for five days before being freeze-dried. To create a GelMA solution, the freeze-dried GelMA was dissolved at different concentrations (w/v) in pure water with 1% (w/v) of an photoinitiator which is sensitive to ultraviolet (UV) light, 2hydroxy-1-(4-(hydroxyethoxy)phenyl)-2-methyl-1-propanone (Irgacure 2959, Sigma Aldrich). 10%, 15%, and 20% (w/v) GelMA concentrations were used for this study.

PEGDA solutions were made by combining 10% (v/v) poly (ethylene glycol) diacrylate (PEG-DA, Mn 700) at room temperature with 1% (w/v) 2hydroxy-1-(4-(hydroxyethoxy)phenyl)-2-methyl-1-propanone (Irgacure 2959, Sigma Aldrich) dissolved in PBS. The solution was kept at 65° C. until fully dissolved. For samples with GelMA in addition to the PEGDA, the desired concentration of GelMA was dissolved in the PEGDA solution. For this study, 0%, 5%, and 10% (w/v) GelMA were used with the PEGDA.

### Sample Preparation

Samples were prepared by crosslinking the hydrogel solutions layer by layer. For rheological testing, samples were made by pipetting one layer of solution into a 35-mm petri dish, placing a cylindrical mold (20 mm diameter) on top of the solution, and exposing the solution to UV light (365 nm, 28 mW cm⁻²). The cured hydrogels were left in the mold during swelling to minimize changes in sample diameter. Due to differences in the thickness of GelMA and PEGDA hydrogels after swelling, the amount of solution used to create these samples was varied: 1.2 mL for the GelMA hydrogels and the PEGDA hydrogel with 10% GelMA, 1.4 mL for the PEGDA hydrogel with 5% GelMA, and 2 mL for the PEGDA-only hydrogel. This resulted in samples of approximately the same thickness.

The sample preparation was modified slightly for acoustic testing; three additional layers of solution were added to the mold and exposed to UV light, one by one. The initial amount of solution was scaled by the area of the cylinder to determine the amount pipetted for each layer. The UV light curing times were varied in 30 s increments from 30 s to 120 s for the 15% GelMA samples. For all other samples, a curing time of 60 s was used for each layer. A summary of the sample groups is given in Table 1.

TABLE 1
Hydrogel sample groups

| | | Hydrogel | UV light |
|---|---|---|---|
| | Hydrogel type | concentration | curing time |
| Group 1: | GelMA | 10% | 60 s |
| Varying hydrogel | | 15% | |
| concentration | | 20% | |
| Group 2: | PEGDA | 10% | 60 s |
| Varying hydrogel | PEGDA + GelMA | 10% + 5% | |
| type | PEGDA + GelMA | 10% + 10% | |
| Group 3: | GelMA | 15% | 30 s |
| Varying UV | | | 60 s |
| light curing time | | | 90 s |
| | | | 120 s |

### Acoustic Measurements

Referring to FIGS. 5(a), (b), one illustrative embodiment of the disclosure is shown. The acoustic setup comprises a processing device such as a computer, an oscilloscope, a pulser-receiver, an attenuator, and the characterization assembly 100 which includes a 2.25 MHz flat transducer 150.

The transducer face was submerged in a water-filled chamber and kept parallel to the bottom of the chamber. Three control signals were taken. Next, a hydrogel was placed in the chamber, directly below the transducer face. Three test signals were taken. By comparing the time of the test signals to the time of the control signals, the speed of sound through the hydrogel was found. By comparing the amplitude of the test signals to the amplitude of the control signals, the attenuation was found.

### Rheology Measurements

Steady shear rheological tests (n=3) were performed using a rotational rheometer (DHR-2, TA Instruments) with a parallel plate geometry (0.75 mm measuring distance). Stainless steel crosshatched plates were selected to minimize slip (20.0 mm upper plate, 40.0 mm lower Peltier Plate). The setup is shown in FIG. 1. Each measurement was performed with a new sample, and samples were patted dry before loading to remove excess water. After loading, samples were conditioned for 60 s at 27° C. A logarithmic flow sweep was then performed for shear rates from 0.1 s⁻¹ to 1,000.0 s⁻¹, and 41 data points were collected. TRIOS Software (TA Instruments) was used to record the shear stress at every shear rate. The ratio of shear stress to shear rate was found for each data point, resulting in the apparent viscosity of each sample as a function of shear rate.

### Compression Measurements

A universal mechanical test machine (Applied Test Systems, Butler, PA) was used to uniaxially compress each sample. The samples were measured using digital calipers (sensitivity 0.01 mm) then tested using a 100 N load cell at a crosshead speed of 1 mm min⁻¹. Young's modulus was calculated from the linear region of the resultant stress-strain curves.

FIG. 1 shows the viscoelasticity characterization methods and illustrative experimental data. The data shown are for GelMA hydrogel samples (60 s curing time) of varying concentrations: 10% (yellow), 15% (red), and 20% (blue). FIG. 1(a) shows a rotational rheometer setup showing a hydrogel sample between stainless steel crosshatched plates. FIG. 1(b) shows flow sweeps obtained using the rotational rheometer. The viscosity of the samples are shown as a function of shear rate. Shear thinning is exhibited by all three samples. FIG. 1(c) shows mechanical testing setup showing a hydrogel sample as it is uniaxially compressed. FIG. 1(d) shows stress-strain curves obtained from the compression test. Young's modulus can be found as the slope of the linear region of each curve. FIG. 1(e) shows the acoustic setup for the pulse echo ultrasound tests. A transducer (2.25 MHz center frequency) is submerged in a chamber filled with water. A hydrogel sample is at the bottom of the chamber. FIG. 1(f), 1(j) shows an example of the signals received by the transducer. The test signal (which traveled through the water and the hydrogel) arrives earlier and has a smaller amplitude than the control signal (which traveled through the water alone) due to sound traveling faster in the hydrogel. FIG. 1(g) shows an acoustic attenuation coefficients for 1.25-3.75 MHz, calculated using the difference in amplitude between the control and test signals. Using these values, the viscosity of each sample can be calculated as a function of frequency. FIG. 1(h) shows the speed of sound through each hydrogel. These values can be used to calculate the bulk moduli.

FIG. 2 shows the elastic moduli of the samples. In FIG. 2(a), the bulk modulus of each sample was calculated using hydrogel sound speeds from pulse echo ultrasound tests. In FIG. 2(b), Young's modulus of each sample was calculated from compression tests.

FIG. 3 shows a comparison of the viscosity values measured using rotational rheology (blue) and the viscosity values calculated from pulse echo ultrasound (red). Individual viscosity values with their standard deviations are plotted at their corresponding frequencies. The average of the measured viscosities (dashed blue line) and the average of the calculated viscosities (dashed red line) are also plotted. FIGS. 3(a), (b), and (c) show how both viscosities increase with GelMA concentration. FIGS. 3(d), (e), and (f) show how both viscosities increase with the addition of GelMA to PEGDA hydrogels. FIGS. 3(g), (h), and (i) show how both viscosities increase with curing time between 30 s and 90 s. FIG. 3(j) shows how both viscosities decrease between 90 s and 120 s curing time.

FIG. 4 is a comparison of the viscosity trends measured using rotational rheometry (blue) and the viscosity values calculated from pulse echo ultrasound (red). FIG. 4(a) shows the viscosity increased with each increase in GelMA concentration, with the pulse echo ultrasound values falling within the standard deviation of the rotational rheometry values. FIG. 4(b) shows the viscosity increased as GelMA was added to PEGDA hydrogels, with the pulse echo ultrasound values closely following the rotational rheometry values. FIG. 4(c) shows the viscosity increased as the curing time of the GelMA hydrogels increased from 30 s to 90 s, then the viscosity decreased as the curing time increased from 90 s to 120 s. The standard deviations of the viscosities from rotational rheometry and pulse echo ultrasound overlap.

Thus, a low-cost device (ultrasound transducer connected to a small electrical system) is provided that directly measures the viscosity and elasticity of a hydrogel sample. We also provide a computer program that processes the ultrasound data and performs the viscosity and elasticity calculations. With minimal inputs, a non-invasive, non-destructive material characterization would be performed. This would be particularly applicable in the field of tissue engineering. In certain embodiments, a single-transducer longitudinal ultrasound characterization setup/technique is provided. The system is a non-destructive, non-invasive method for determining the viscosity of a hydrogel (in sharp contrast to the traditional rheometry method which requires direct contact with the hydrogel).

### Speed of Sound and Attenuation Coefficient Measurements

A time-of-flight approach was used to obtain the speed of sound (step 236) through each hydrogel. Using the setup shown in FIG. 5, signals were recorded when the ultrasound pulse traveled through water alone and when it traveled through water as well as a hydrogel. FIG. 1(j) shows a representative voltage-time signal with and without the hydrogel. The ultrasound pulse trace comparison with and without a hydrogel in the path. A 2.25 MHz flat transducer was used as the source.

The temporal shift (Δt) between these signals was found using cross-correlation. The speed of sound (n=9) for each hydrogel is

$${c}_{h} = {( \frac{1}{{c}_{w}} - \frac{Δ t}{2 d} )}^{- 1} , \qquad ( 3 )$$

where cₕ is the speed of sound through the hydrogel, c_{w} is the known speed of sound through pure water at room temperature, and d is the thickness of the hydrogel. The speed of sound through pure water was taken to be 1483 m s. The thickness of each hydrogel was measured using a digital caliper with a sensitivity of 0.01 mm, and the average of three measurements was used for each calculation.

The attenuation (step 238) of sound through the hydrogels was measured using the same experimental procedure, obtaining signals for pulses traveling through water alone and for those traveling through water and hydrogel. The FFT of each signal was computed to obtain the received signal amplitude as a function of frequency f. The attenuation coefficient of water, α_{w}(f), is related to the FFT of the received signal amplitude, A_{w}(f), as follows

$${A}_{w} ( f ) = {U}_{0} ( f ) {e}^{- 2 {α}_{w} ( f ) l} , \qquad ( 4 )$$

where U₀(f) is the initial amplitude of the received signal and l is the path length of the signal. Similarly, when a hydrogel is placed in the path the attenuation of the signal over the entire path, αₛ(f), is related to the FFT of the received signal amplitude, Aₛ(f), as follows

$${A}_{s} ( f ) = {U}_{0} ( f ) {{e}^{- 2 {α}_{w} ( f ) ( l - d )} ( {T}_{I , II} {T}_{II , I} )}^{2} {e}^{- 2 {α}_{s} ( f ) d} , \qquad ( 5 )$$

where d is the thickness of the hydrogel, T_{I,II} is the transmission coefficient from water to the hydrogel, and T_{II,I} is the transmission coefficient from the hydrogel to water. The transmission coefficients are determined by the acoustic impedance of water,

$${Z}_{w} = {ρ}_{w} {c}_{w} , \qquad ( 6 )$$

where the density of water is ρ_{w} and the speed of sound through water is c_{w}, and the acoustic impedance of the hydrogel,

$${Z}_{h} = {ρ}_{h} {c}_{h} . \qquad ( 7 )$$

where the density of the hydrogel is ρₕ and the speed of sound through the hydrogel is cₕ. The transmission coefficients are

$${T}_{I , II} = \frac{2 {Z}_{h}}{{Z}_{w} + {Z}_{h}} \qquad ( 8 )$$

$$and$$

$${T}_{II , I} = \frac{2 {Z}_{w}}{{Z}_{w} + {Z}_{h}} , \qquad ( 9 )$$

The attenuation coefficient (n=5) of each hydrogel, αₕ(f), was found by subtracting the attenuation coefficient values through water from the attenuation coefficient values through water and the hydrogel

$${α}_{h} ( f ) = {α}_{s} ( f ) - {α}_{w} ( f ) = \frac{1}{2 d} ln [ \frac{{A}_{w} ( f )}{{A}_{s} ( f )} ] + \frac{1}{d} ln ( {T}_{I , II} {T}_{II , I} ) . \qquad ( 10 )$$

The following conversion is used to obtain attenuation coefficient values in units of dB cm⁻¹:

$${α}_{h} ( f ) = \frac{20}{d} {log}_{10} [ \frac{{A}_{w} ( f )}{{A}_{s} ( f )} ] + \frac{20}{d} {log}_{10} ( {T}_{I , II} {T}_{II , I} ) . \qquad ( 11 )$$

It is noted that biomedical samples scatter acoustic signals due to the inhomogeneity of the sample and the acoustic signal reflecting off of the various sample interfaces. In the present system 5, however, hydrogel samples are homogeneous. Acoustic wavelength is mm while polymers are nanometer. The scatter is due to inhomogeneity of the sample, as illustrated in FIG. 1(j).

The following references are hereby incorporated by reference. 1. Wells, P. N. T. & Liang, H.-D. Medical ultrasound: imaging of soft tissue strain and elasticity. J. R. Soc. Interface. 8, 1521-1549 (2011). 2. Spicer, C. D. Hydrogel scaffolds for tissue engineering: the importance of polymer choice. Polym. Chem. 11, 184-219 (2020). 3. Duck, F. A. Physical properties of tissue: a comprehensive reference book. (Academic Press, 1990). 4. Stokes, G. G. Mathematical and Physical Papers vol. 1. (Cambridge University Press, 2009). doi:10.1017/CB09780511702242. 5. Garrett, S. L. Understanding Acoustics: An Experimentalist's View of Sound and Vibration. (Springer International Publishing, 2020). doi:10.1007/978-3-030-44787-8. 6. Pierce, A. D. Acoustics: An Introduction to Its Physical Principles and Applications. (Springer International Publishing, 2019). doi:10.1007/978-3-030-11214-1. 7. Dukhin, A. S. & Goetz, P. J. Bulk viscosity and compressibility measurement using acoustic spectroscopy. The Journal of Chemical Physics 130, 124519 (2009). 8. Holm, S. Waves with Power-Law Attenuation. (Springer International Publishing, 2019). doi:10.1007/978-3-030-14927-7. 9. Chemical Rubber Company. CRC handbook of chemistry and physics: a ready-reference book of chemical and physical data. (CRC Press, 2003). 10. Hosono, K., Maruyama, H., Ikeda, S. & Arai, F. Heart Surgery Simulator Using Hydrogel Having Heating Temperature Memory Function. Robomech 2018, 1P2-E01 (2018). 11. Tang, N. et al. Thermal Transport in Soft PAAm Hydrogels. Polymers 9, 688 (2017). 12. Girnyk, S. et al. The estimation of elasticity and viscosity of soft tissues in vitro using the data of remote acoustic palpation. Ultrasound in Medicine & Biology 32, 211-219 (2006). 13. Sigrist, R. M. S., Liau, J., Kaffas, A. E., Chammas, M. C. & Willmann, J. K. Ultrasound Elastography: Review of Techniques and Clinical Applications. Theranostics 7, 1303-1329 (2017). 14. Van Den Bulcke, A. I. et al. Structural and Rheological Properties of Methacrylamide Modified Gelatin Hydrogels. Biomacromolecules 1, 31-38 (2000). 15. Osborn, J., Anderson, M. S., Beddingfield, M., Zhang, L. G. & Sarkar, K. Acoustic Droplet Vaporization of Perfluorocarbon Droplets in 3D-Printable Gelatin Methacrylate Scaffolds. Ultrasound in Medicine & Biology (2021) doi:10.1016/j.ultrasmedbio.2021.07.016. 16. Zhou, X. et al. Three-Dimensional-Bioprinted Dopamine-Based Matrix for Promoting Neural Regeneration. ACS Appl. Mater. Interfaces 10, 8993-9001 (2018).

The processing device, such as the computer 160, can be, for instance, a computer, personal computer (PC), server or mainframe computer, or more generally a computing device, processor, application specific integrated circuits (ASIC), or controller. The processing device can be provided with, or be in communication with, one or more of a wide variety of components or subsystems including, for example, a co-processor, register, data processing devices and subsystems, wired or wireless communication links, user-actuated (e.g., voice or touch actuated) input devices (such as touch screen, keyboard, mouse) for user control or input, monitors for displaying information to the user, and/or storage device(s) such as memory, RAM, ROM, DVD, CD-ROM, analog or digital memory, flash drive, database, computer-readable media, floppy drives/disks, and/or hard drive/disks. All or parts of the system, processes, and/or data utilized in the system of the disclosure can be stored on or read from the storage device(s). The storage device(s) can have stored thereon machine executable instructions for performing the processes of the disclosure. The processing device can execute software that can be stored on the storage device. Unless indicated otherwise, the process is preferably implemented automatically by the processor substantially in real time without delay.

It is further noted that the drawings may illustrate and the description and claims may use several geometric or relational terms and directional or positioning terms, such as elongated, perpendicular, orthogonal, vertical, horizontal, flat, top, bottom, left, right, interior, exterior, distal, and proximal. Those terms are merely for convenience to facilitate the description based on the embodiments shown in the figures, and are not intended to limit the disclosure. Thus, it should be recognized that the disclosure can be described in other ways without those geometric, relational, directional or positioning terms. In addition, the geometric or relational terms may not be exact. For instance, walls or surfaces may not be exactly flat, perpendicular or parallel to one another but still be considered to be substantially perpendicular or parallel because of, for example, roughness of surfaces, tolerances allowed in manufacturing, etc. And, other suitable geometries and relationships can be provided without departing from the spirit and scope of the disclosure.

As used herein, when an element or feature is described as being “configured,” that element or feature is structurally arranged or formed to accomplish the stated purpose. As used with respect to a processing device (e.g., computer), the term “configured” means that the processing device is structurally arranged or ordered (e.g., by supplying, arranging or connecting a specific set of internal or external components or modules, for example that perform certain operations) to accomplish the stated purpose or task.

The description and drawings of the present disclosure provided in the paper should be considered as illustrative only of the principles of the disclosure. The disclosure may be configured in a variety of ways and is not intended to be limited by the disclosed embodiment. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A system for analyzing a hydrogel sample, the system comprising:
a plate configured to retain the hydrogel sample;
an ultrasound transmitter configured to propagate an ultrasound signal toward the hydrogel sample retained at said plate;
an ultrasound detector configured to receive a reflected ultrasound signal reflected by said plate and passing through the hydrogel sample; and
a processing device configured to determine a property of the hydrogel sample based on the reflected ultrasound signal, wherein the property comprises viscosity.

2. The system of claim 1, wherein the property further comprises elasticity of the hydrogel sample.

3. The system of claim 1, further comprising identifying the hydrogel sample based on said determined property.

4. The system of claim 1, wherein said processing device is configured to determine the speed of the ultrasound signal through the hydrogel sample.

5. The system of claim 1, wherein said ultrasound transmitter and said ultrasound detector comprise a transducer.

6. The system of claim 5, wherein said transducer comprises a single transducer.

7. The system of claim 1, wherein the hydrogel sample is not a liquid.

8. The system of claim 1, wherein the ultrasound signal is not a shear wave.

9. The system of claim 1, wherein the ultrasound signal is not destructive to the hydrogel sample.

10. The system of claim 1, wherein the ultrasound signal is an acoustic frequency from 1.5-3 MHz.

11. The system of claim 1, wherein the ultrasound signal is an acoustic frequency from 0.5-5 MHz.

12. The system of claim 1, wherein said processing device determines a viscosity of the hydrogel sample as:
$$α = \frac{2 {ηω}^{2}}{3 ρ {c}^{3}} ,$$
where α is the acoustic attenuation coefficient at a specific selected angular frequency ω corresponding to dynamic viscosity η of the hydrogel sample, c is the speed of sound.

13. The system of claim 1, wherein the ultrasound signal is non-invasive.

14. The system of claim 1, further comprising a container having a bottom comprising said plate and the hydrogel sample, said container further receiving a liquid.

15. The system of claim 14, said container further receiving a liquid medium, at least a portion of said ultrasound transmitter, and at least a portion of said ultrasound detector,
wherein said ultrasound transmitter and said ultrasound detector are at least partially received in said liquid medium to propagate the ultrasound signal and the reflected ultrasound signal between said ultrasound transmitter, said ultrasound detector, said plate and the hydrogel sample.

16. The system of claim 1, wherein the hydrogel sample comprises a soft biogel.

17. The system of claim 1, wherein the hydrogel sample comprises gelatin methacrylate (GelMA) or poly-(ethylene glycol)-diacrylate (PEGDA) hydrogels or polymeric hydrogels.

18. The system of claim 1, wherein the hydrogel comprises or a biomaterial sample.

19. The system of claim 18, wherein the biomaterial sample comprises a tissue.

20. A method for analyzing a hydrogel sample, comprising:
passing an ultrasound signal through the hydrogel sample;
detecting a reflected ultrasound signal from the hydrogel sample; and
determining a property of the hydrogel sample based on the reflected ultrasound signal.
